(19) United States
(12) Patent Application Publication
(10) Pub. No.: US 2022/0130535 A1
(43) Pub. Date: Apr. 28, 2022

(54) SYSTEM AND METHOD FOR REMOTE AUTHENTICATED TESTING APPLICATIONS

(71) Applicant: S-Square Systems, LLC, South Barrington, IL (US)

(72) Inventors: Arun Sobti, South Barrington, IL (US); Raj Panchal, South Barrington, IL (US)

(21) Appl. No.: 17/499,449

(22) Filed: Oct. 12, 2021

Related U.S. Application Data
(60) Provisional application No. 63/090,588, filed on Oct. 12, 2020.

(51) Int. Cl. G16H 40/67; G16H 50/20; G16H 10/40

(52) U.S. Cl. CPC G16H 40/67; G16H 10/40; G16H 50/20; H04L 63/04

(57) ABSTRACT

The present disclosure provides a system and method for facilitating authenticated remote testing, including conducting the test and certifying the results of the conducted test. The system includes a means to securely deliver a standardised testing kit to a patient, verify validity and authenticate the testing kit, while ensuring that the testing kit is not compromised. The patient can self-administer the test under the supervision (if required) of a medical practitioner. For any remote testing location, the system provides a secure means to deliver the sample to the testing location. The results of the testing at the remote testing location can then be communicated to both the patient and the medical service provider in the form of a certificate if required. The patient can receive a remote testing unit at home or purchase one at a kiosk, and a method for administering the test.

### CROSS-REFERENCE TO RELATED APPLICATIONS

This application claims the benefit of Provisional U.S. Patent Application No. 63/090,588 filed Oct. 12, 2020, entitled “SYSTEM AND METHOD FOR REMOTE, AUTHENTICATED TESTING APPLICATIONS”, the entire content and disclosure of which, both express and implied, is incorporated herein by reference.

### TECHNICAL FIELD

The present disclosure relates generally to the field of authentication and certification of diagnostic testing process and results of the testing obtained in telemedicine or in testing centers. In particular, the present disclosure relates to authentication and certification of testing in centers to prevent spread of any contagion amongst people present in the testing population. Further, the present disclosure relates to an authenticated and secure exchange of health data between a patient and a medical service provider.

### BACKGROUND

Medical testing and the parameters revealed during the testing play a vital role in diagnosing conditions of a patient. There exist a large number of tests which can be classified as self-administering tests where the patient or a care provider of the patient can administer the test to report the results or to send a sample collected during the test to a testing location. Such tests can include swabs, simple blood collection and reading of parameters such as blood pressure, pulse rate, blood oxygen etc.

Testing kits can be directly delivered to the patient. However, during delivery, there is a chance that the testing kits may be damaged, be expired in validity or indeed, a wrong testing kit is provided. Further, upon receipt of the testing kit, there is a chance that the test may be incorrectly administered, leading to results that may not be accurate and consequently leading to diagnoses that are faulty.

Often, during a season or period of contagious illnesses, a contagion may spread from persons infected to those who are not. A true and tested approach to limit spread is to isolate the infected from the uninfected. However, it is imperative that any test administered for detection of the infection be from an authorized source, and the results be certified and authenticated by a competent authority.

There is, therefore, a requirement in the art for a remote health application to facilitate remote administration of tests and procedures, where the patient can be interactively guided to administer the tests and procedures under strict supervision of a medical service provider. Additionally, there is a requirement for the results obtained from the tests or procedures to be certified and authenticated by a competent authority.

### SUMMARY

An example embodiment of the present disclosure pertains to a self-administering testing system for checking health conditions of a first user. The system may include a processor; a probe mechanism coupled to the processor, the probe mechanism to: extract a sample comprising a body tissue of a first user, the body tissue pertaining to a site wherein medical examination is required for the first user.

In an example embodiment, a sensor module coupled to the processor, the sensor module to: extract a set of signals pertaining to health conditions of a user. The system further includes a data capturer coupled to the processor to capture information pertaining to the first user, and a data lake coupled to the processor to store the captured information.

In an example embodiment, a machine learning engine coupled to the processor to: examine the sample extracted from the probe mechanism; extract a first set of attributes pertaining to the examined sample; extract a second set of attributes from the set of signals extracted by the sensor module; correlate the first and second set of attributes to determine a third set of attributes; store the third set of attributes in the data lake; and transmit the third set of attributes through a communication module associated with a first computing device associated with the first user and a second computing device associated with a second user.

An example embodiment of the present disclosure pertains to a self-administering testing device The device may include a processor; a probe mechanism coupled to the processor, the probe mechanism to: extract a sample comprising a body tissue of a first user, the body tissue pertaining to a site wherein medical examination is required for the first user. The device may further include a sensor module coupled to the processor, the sensor module to: extract a set of signals pertaining to health conditions of a user. The device further includes a data capturer coupled to the processor to capture information pertaining to the first user, and a data lake coupled to the processor to store the captured information. The device may further include a machine learning engine coupled to the processor to: examine the sample extracted from the probe mechanism; extract a first set of attributes pertaining to the examined sample; extract a second set of attributes from the set of signals extracted by the sensor module; correlate the first and second set of attributes to determine a third set of attributes; store the third set of attributes in the data lake; and transmit the third set of attributes through a communication module associated with a first computing device associated with the first user and a second computing device associated with a second user.

The present disclosure further pertains to a non-transitory computer readable medium comprising machine executable instructions that are executable by a processor. Upon such execution of the machine executable instructions, the processor may examine a sample extracted from a probe mechanism, wherein the sample comprises a body tissue of a first user, the body tissue pertaining to a site wherein medical examination is required for the first user; extract a first set of attributes pertaining to the examined sample; extract a second set of attributes from a set of signals extracted by a sensor module; correlate the first and second set of attributes to determine a third set of attributes; store the third set of attributes in a data lake; and transmit the third set of attributes through a communication module associated with a first computing device associated with the first user and a second computing device associated with a second user.

### BRIEF DESCRIPTION OF DRAWINGS

Features of the present disclosure are illustrated by way of examples shown in the following figures. In the following figures, like numerals indicate like elements, in which:

FIG. 1 illustrates an example representation of a system for facilitating remote diagnostic testing, in accordance with an embodiment of the present disclosure.

FIG. 2 illustrate an example representation of an apparatus for remote health applications that can be used to facilitate remote diagnostic testing, in accordance with an embodiment of the present disclosure.

FIG. 3A illustrates an example top-level schematic representation of a system for remote health applications that can be used to facilitate remote diagnostic testing, in accordance with an embodiment of the present disclosure.

FIGS. 3B and 3C illustrate example architectures for a system for remote health applications that can be used to facilitate remote diagnostic testing, in accordance with an embodiment of the present disclosure.

FIGS. 4A and 4B illustrate example flow diagrams for a process for facilitating remote diagnostic testing, in accordance with an embodiment of the present disclosure.

FIG. 5 illustrates an example representation of a system for facilitating remote diagnostic testing for a patient where the patient is required to visit a remote testing location, in accordance with an embodiment of the present disclosure.

FIG. 6 illustrates a hardware platform for implementation of the system, according to an example embodiment of the present disclosure.

### DETAILED DESCRIPTION

For simplicity and illustrative purposes, the present disclosure is described by referring mainly to examples thereof. The examples of the present disclosure described herein may be used together in different combinations. In the following description, details are set forth in order to provide an understanding of the present disclosure. It will be readily apparent however, that the present disclosure may be practiced without limitation to all these details. Also, throughout the present disclosure, the terms “a” and “an” are intended to denote at least one of a particular element. As used herein, the term “includes” means includes but not limited to, the term “including” means including but not limited to. The term “based on” means based at least in part on.

### 1. Overview

The present disclosure relates generally to the field of authentication and certification of diagnostic testing process and results of the testing obtained in telemedicine or in testing centers. In particular, the present disclosure relates to authentication and certification of testing in centers to prevent spread of any contagion amongst people present in the testing center. Further, the present disclosure relates to an authenticated and secure exchange of health data between a patient and a medical service provider.

### 2. System Description

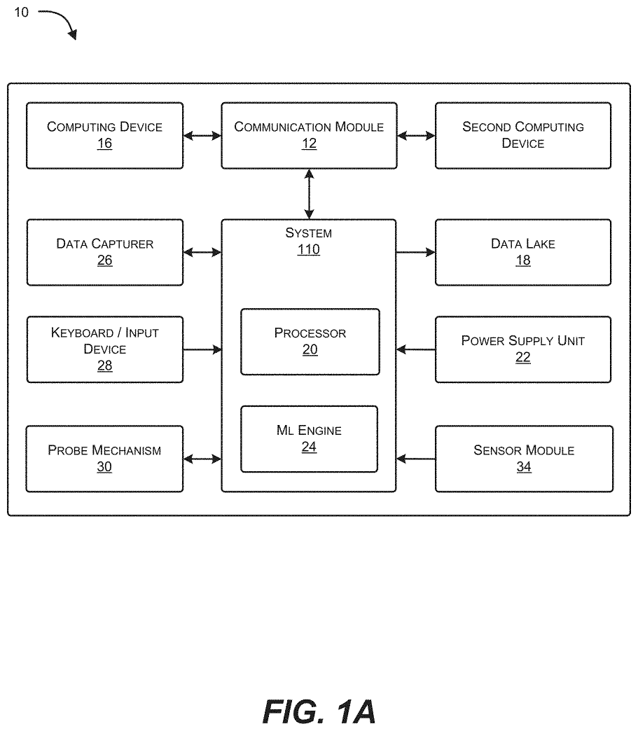

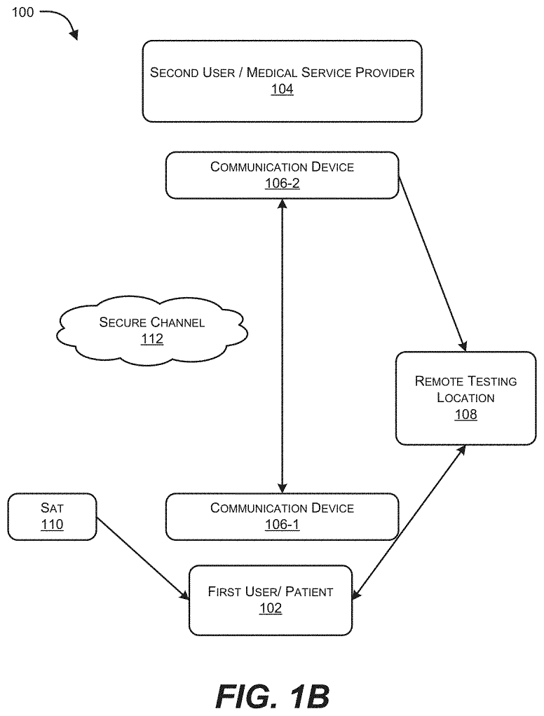

FIG. 1A illustrates a self-administering testing system representation 10, according to an example embodiment of the present disclosure while FIG. 1B illustrates a representation of a system 100 for facilitating remote diagnostic testing, in accordance with an example embodiment of the present disclosure. As illustrated in FIG. 1A, in an example embodiment, a self-administering testing (SAT) system 110 (interchangeably referred to as the system 110 or SAT 110 or device 110 or apparatus 110 or the SAT kit 110 hereinafter) may include a processor 20; a probe mechanism 30, a sensor module 34, a data capturer 26, and a data lake 18.

In an example embodiment, the probe mechanism 30 may be coupled to the processor 20 to extract a sample comprising a body tissue of a first user 102 (Ref. FIG. 1B), the body tissue pertaining to a site wherein medical examination may be required for the first user 102.

In another example embodiment, the sensor module 34 coupled to the processor 20, the sensor module 34 to extract a set of signals pertaining to health conditions of a user. The data capturer 26 coupled to the processor 20 to capture information pertaining to the first user 102 while the data lake 18 coupled to the processor 20 to store the captured information.

In an example embodiment, the SAT 110 may be equipped with a machine learning (ML) engine 24 coupled to the processor 20 to examine the sample extracted from the probe mechanism 30 extract a first set of attributes pertaining to the examined sample; extract a second set of attributes from the set of signals extracted by the sensor module 34; correlate the first and second set of attributes to determine a third set of attributes; store the third set of attributes in the data lake 18; and transmit the third set of attributes through a communication module 12 associated with a first computing device 16 associated with the first user 102 and a second computing device 14 associated with a second user 104.

In a way of example, and not as a limitation, the first user 102 may be a patient or any person needing a medical report on any health condition. The second user 104 may be a doctor, a nurse or any medical service provider.

In an example embodiment, the system 110 may further include a verification and authentication module (not shown in FIG. 1A) that may verify and authenticate the system 110 based on a set of predefined instructions such as per guidelines established for the SAT test. The verification and authentication module may further check expiration validity, type of the system, and a visual recognition mechanism may be coupled to the authentication and verification module to visually recognize any or a combination of a predetermined pattern, timestamp for delivery and receipt of the system, wherein the time stamp may be recorded on the data 18 lake. In a way of example and not as a limitation, the visual recognition mechanism may be a camera, a webcam, a scanner and the like.

In an example embodiment, the system 110 may be configured to obtain a specific registration data based on a request from an unregistered first user through respective first computing device 16. Login credentials may be generated based on acknowledgement of a request and verification of the registration data. The first user 102 may enter the generated login credentials to access the system 110 to obtain the information service associated with the first user 102.

Alternatively, the system 110 may be configured to obtain a registration data based on a request from an unregistered second user through a respective second computing device 14. Login credentials may be generated based on acknowledgement of a request and verification of the registration data. The second user 104 may enter the generated login credentials to access the system 110 to obtain the information service associated with the first user 102.

In an example embodiment, on successful authentication, the system may further provide for remote operation by the second user 104 of the sensor module coupled to the system, including the authentication and instruction to transmit information through a set of audio, video or textual messages through the respective first computing device 16 associated with the first user 102 and the second computing device 14 associated with the second user 104 or through a communication module 12 operatively coupled to the system 110.

In an example embodiment, the communication module 12 may provide communication between the first computing device 16 associated with the first user 102 and the second computing device 14 associated the second user 104 adapted for visual and audio communications. The communication module may be configured with security protocols to facilitate communication between the first user 102 and the second user 104.

In an example embodiment, the sample extracted by the probe mechanism 30 may be collected and securely stored and sealed under supervision in one or more containers associated with the system. The one or more containers may be marked with identification markers pertaining to identity of the first user 102, the nature of the test and the identity of the second user 104 and the time stamp for administration of the system.

In an example embodiment, a result associated with examination of the third set of attributes may be provided in the form of a certificate indicating that the result provided is authenticated by a competent authority. The certificate may include the predetermined pattern that upon scanning by a scanner, a set of details included in the certificate may be available to any user or entity performing the scan. The set of details may include the information associated with the first user 102, any contagious disease suffering by the first user 102 or a combination thereof.

In an example embodiment, the SAT 110 may be customized to suit the testing needs of the first user. The SAT may be further configured to communicate with a predetermined entity associated with providing and delivering the SAT. The SAT may be with tamper-proof seals and weather-proof seals with a predefined size to fit inside and get delivered from a predetermined kiosk.

In a way of example and not as a limitation, the system 110 can include an approach to receive, by the patient at a location where the patient is situated, and administer, by the patient or a care provider situated at the location of the patient, a self-administering test (herein, abbreviated as “SAT”) kit as per guidelines established for the SAT test. The administration of the SAT can occur under the supervision of a trained medical service provider 104 with the medical service provider being located remotely. For purposes of authentication, the trained medical service provider may be considered as a trusted authority (TA).

In another example embodiment, the system 110 also includes a means to verify and authenticate the received SAT kit as being an approved and appropriate testing kit that is still within its validity date, according to instructions issued pertaining to the testing kit, either by the medical service provider 104, the TA or the patient 102. Inclusion of the TA adds to authentication of delivery and administration of the SAT.

In a way of example and not as a limitation, any communication between the patient 102 and the medical service provider 104 can occur through the communication module that can include an apparatus adapted for visual and audio communications. The apparatus can be provided with each of the patient and the medical service provide in order that real-time communication between the medical service provider and the patient can occur. At least two channels of video communications may be allowed simultaneously—one for communications (audio-video) between the patient 102 and the medical care provider 104, and the second focused on the test being administered. An example device can be any communications device with an interface device coupled to it. The communications device 106-1, 106-2 may be a proprietary device, or may be any device already available in the art. While the apparatus is not within the gamut of the present disclosure, an example apparatus is described in subsequent sections of this application.

In a way of example and not as a limitation, the apparatus can have security protocols that facilitate all communications between the patient 102 and the medical service provider 104 to be compliant with established protocols. An example of such a protocol can be Health Insurance Portability and Accountability Act (HIPAA) but not limited to the like.

In another example embodiment, the apparatus can include a function for requesting and procuring standardized SAT kits from vendors, with the authenticity of the vendors having been previously verified.

In another example embodiment, the requesting and procurement of the SAT kit can be made by the medical service provider 104 or by the patient 102.

In another example embodiment, the requested SAT kit can then be delivered to the patient at the location 108 of the patient 102, by any delivery services. In a way of example and not as a limitation, the delivery of the SAT kit 110 can have mechanisms to ensure that the testing kit is not compromised in any way, which can include the use of tamper-proof seals and weather-proof seals on the testing kits.

In another example embodiment, the apparatus provided with the patient can have mechanisms to verify the authenticity, expiration validity and type of SAT kit delivered to the patient. The mechanism can include visual recognition of the SAT kit, the visual recognition being any pattern recognition system such as barcodes, QR codes etc. The mechanism can also include a time stamp for delivery and receipt of the SAT kit, where the time stamp may be recorded on a server.

In another example embodiment, once verified that the testing kit is pristine, the medical service provider 104 can instruct the administration of the SAT kit.

In the case the SAT kit is one that collects a sample material from the patient, the sample, upon being collected is securely stored and sealed under visual supervision in one or more containers provided in the testing kit. The one or more containers can have identification markers pertaining to identity of the patient, the nature of the test and the identity of the medical service provider and time stamp for administration of the SAT so that the sample material can be tracked accurately. The sample can then be transported by the delivery service. Mechanisms similar to the one described previously can be employed to ensure that the sample is not compromised in any way along the way.

In another example embodiment, the SAT kit can be one where a result is immediately observed. The result can be communicated to the medical service provider 104 through the secure channels 112 of communication established between the apparatus provided with the patient and the apparatus provided with the medical service provider.

In another example embodiment, the result communicated with the patient can include a certificate indicating that the result provided is authenticated by a competent authority. The certification can imply that the result provided may be considered valid by any one or more entities affiliated to or associated with the competent authority. The certificate can include details of the patient and the test such as name of patient, identification markers pertaining to identity of the patient, the nature of the test and the identity of the medical service provider, data and time of the test and the results.

In an example embodiment, the certificate can include a QR code or barcode that may be scanned by a scanner, whereupon the details included in the certificate may be available to any person or entity performing the scan. In another example embodiment, the certificate can include a serial number.

In an example implementation, the SAT may relate to any contagious disease spread by a contagion. A result of the test indicating a presence or an absence of infection may be coded into the QR code or barcode on the certificate, and where a scan of the code may additionally instruct the entity or persons performing the scan of any subsequent steps to be taken as per an established protocol. For instance, a certified result indicating absence of infection may be used as a gate pass to allow access to persons owning the certified result.

In an example embodiment, the certificate may be provided to the patient in hard copy, or in a soft form as email, test message or any other form of instant messaging. The certificate may also be provided on any dedicated application, or merely inserted into an electronic wallet available on a mobile device such as phone or tablet.

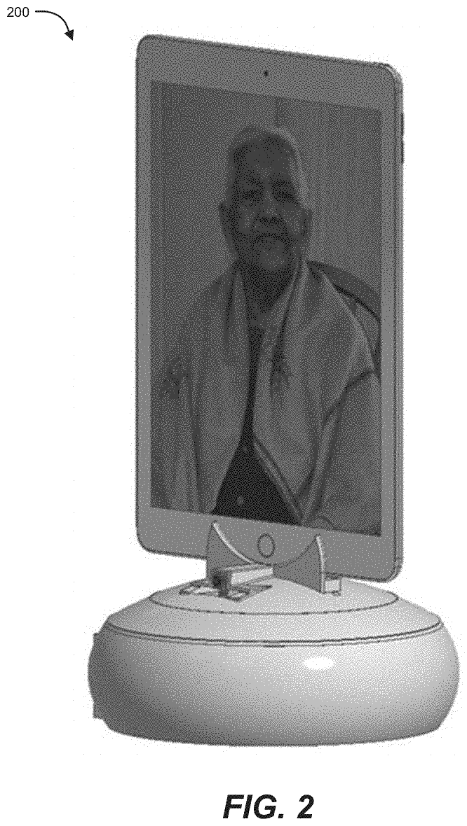

FIG. 2 illustrate an example representation of an apparatus 200 for remote health applications that can be used to facilitate remote diagnostic testing, in accordance with an embodiment of the present disclosure.

In an embodiment, the apparatus 200 can be a dock that is capable of being operatively coupled to an interface device for remote health applications. The digital interface device can be any device capable of audio-video interfacing such as tablet, smartphone, laptop etc. In another example embodiment, the dock can comprise a stand to accommodate interface devices such as a tablet or a smartphone.

In an alternate embodiment, the apparatus 200 can be any communications device capable of audio-visual communications, such as a camera attached to a “stick” (for instance, a FireStick from Amazon), which may then be connected to a television set).

In another example embodiment, the dock (herein, also referred to as “apparatus”, wherein the apparatus may include, without limitations, the dock or any or communications device such as the “stick”) can also be operatively coupled to other input means such as keyboard, mouse etc. The input means can also include a touch-enabled screen on the interface device or on the apparatus.

In another example embodiment, the input/output means can include a voice assistant (VA). The apparatus can be operatively configured with the VA that can be customized for telehealth applications and that can be personalized for the patient or the healthcare provider.

In another example embodiment, the interface device can also be operatively configured with a VA that can be customized for telehealth applications and that can be personalized for the patient or the healthcare provider.

In another example embodiment, the VA may be personalized or customized to be interactive with the patient and the healthcare provider in order to implement processes and procedures necessary for administration and execution of the SAT. The procedures may also involve modifications such as calibration or fine-tuning of an implanted, or explanted devices of the patient to treat conditions such as seizures.

In another example embodiment, the input means can also include a camera, which can be provided with an adjustable pan, tilt and zoom/autofocus functionality for the host apparatus. Here, the camera may be coordinated with the VA to implement processes and procedures necessary for administration and execution of the SAT.

Generally, most smart devices available in the art are provided with a VA. However, the extent to which the VA can be customized and personalized is limited by restrictions placed on them by their respective manufacturers.

For collaborative applications such as telehealth, higher order of personalization is desired to facilitate broader use-cases such as, VA-to-VA collaborations, either locally or remotely. For personalization, within a given platform ecosystem, across platform ecosystems of the collaborating systems or both, a higher order of personalization is desired.

In another example embodiment, the apparatus can include a plurality of ports with pins of different configurations to interface with external devices. The apparatus can also be enabled with wireless technologies to be operatively coupled to external devices. The external devices can include a plurality of health sensors (which may be any or a combination of implanted and explanted devices), input devices such as keyboards, mouse, scanner, biometric device, microphone etc., output devices such as display, speakers, indicator lamps etc. In another example embodiment, the apparatus can also include a charging port for input power.

In another example embodiment, the apparatus can be provided with a stand to accommodate interface devices such as a tablet or a smartphone. The stand can be adjustable in order that the tilt and orientation of the interface device can be adjusted. The pan and tilt orientation can be instrumented leveraging the camera of the docked interface device or that of the apparatus itself in order to track motion and keep the patient in view.

In another example embodiment, the interface device can further be operatively coupled to the apparatus through a port. Any or both of microphone and speaker can be included in the apparatus, operatively coupled to the interface device. In another example embodiment, the apparatus can be configured with a charging outlet to charge the interface device as it is being held in the stand.

In another example embodiment, the apparatus can include wireless connectivity technologies such as Wi-Fi, radio, Bluetooth, mobile internet connectivity etc. through which it can receive and transmit data. The apparatus can be operatively coupled to the external devices through any of the aforementioned wireless communication technologies.

In an example implementation, the apparatus can be integrated with a system for remote health applications such as remote doctor/patient consultation and remote patient monitoring with real-time vital parameters or special conditions of the patients such as non-convulsive seizures.

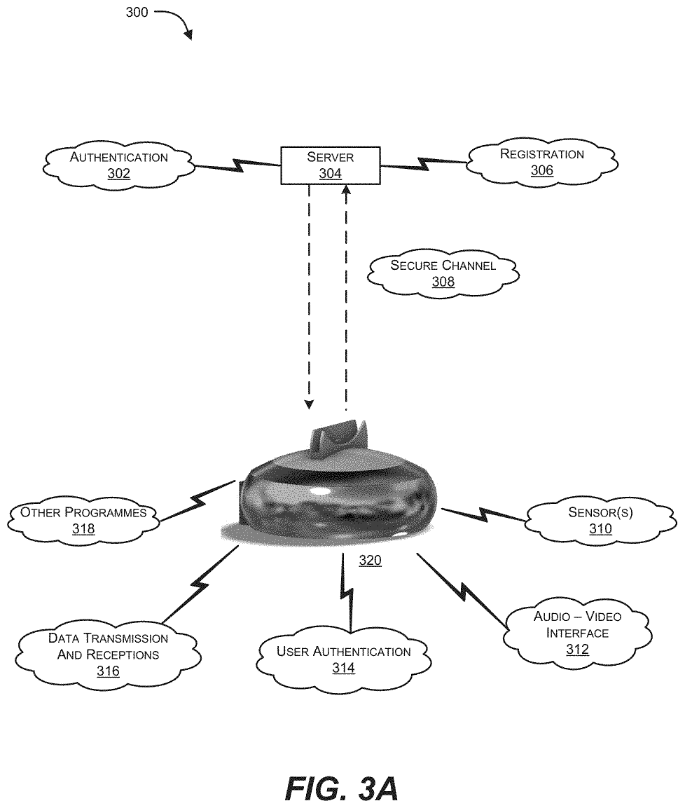

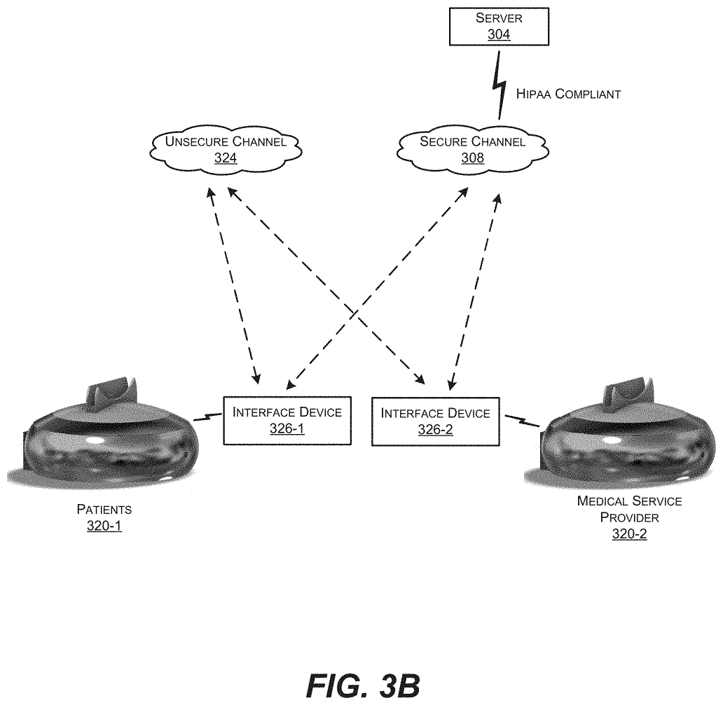

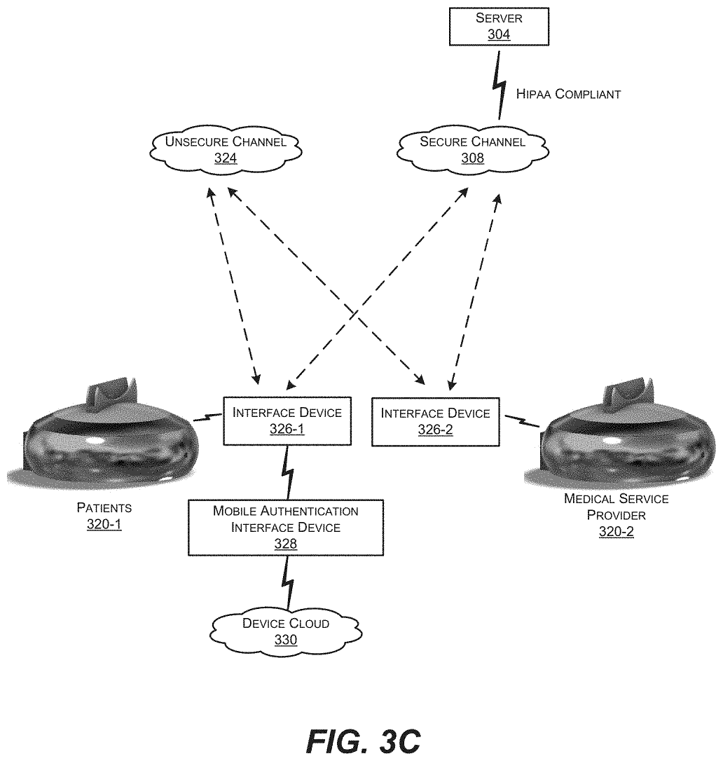

FIG. 3A illustrates an example top-level schematic representation 300 of a system for remote health applications that can be used to facilitate remote diagnostic testing, in accordance with an embodiment of the present disclosure.

In an embodiment, the system can be integrated with the apparatus 320 of the present disclosure to provide remote health applications. The apparatus 320 can comprise one or more processors operatively coupled with a memory, the memory storing instructions to enable remote health applications, which can be executable by the one or more processors.

In another example embodiment, the apparatus can be operatively coupled to a server 304. The server 304 can be configured to receive updates from external devices pertaining to health applications, that can then be transmitted to the apparatus. The channel of communication 308 between the server 304 and the apparatus 320 is made secure. In another example embodiment, the server can be further operatively coupled to other apparatus. Further, the server 304 can be configured to receive updates from external devices pertaining to health applications, that can then be transmitted to the apparatus.

In another example embodiment, the server 304 may be operatively coupled with any one or more authenticated networks such as networks of healthcare organizations, where data from the server 304 may be shared across the one or more authenticated networks 302, either in context of a particular patient's registered account 306 or an artificial intelligence inference engine for statistical analyses.

FIGS. 3B and 3C illustrate example architectures for a system for remote health applications that can be used to facilitate remote diagnostic testing, in accordance with an embodiment of the present disclosure.

In an alternative embodiment, the docked or connected interface device 326 can also be operatively coupled to a server 304.

In another example embodiment, each apparatus 320 can be registered with the server by means of a unique ID. The registration can further be used as any of one or a combination of authenticating means required for transfer of data between the apparatus and the server and between the server and the external devices.

In an example embodiment, the system is configured to enable a patient 320-1 to communicate with a healthcare provider 320-2 via the interface device 326-2. The interface device can be any touch-enabled device available in the art equipped with any available operating system. The communication means can be any as provided by the interface device 326.

In another example embodiment, the system is configured to communicate with the healthcare provider 320-2 using two or more channels of communication. A first channel allows audio-video communication 312 between the healthcare provider 320-2 and the patient 320-1. A second channel 308 allows exchange of data 316 that is construed to be confidential between the patient and the healthcare provider. The confidential information can include, data from the plurality of sensors 310, non-medical data pertaining to the patient such as insurance details etc. In an example embodiment, the second channel 308 is used for exchange of data that is regulated by an established protocol such as Health Insurance Portability and Accountability Act (HIPAA).

In another example embodiment, the system is configured to use both channels simultaneously to exchange data between the patient and the healthcare provider. However, exchange is possible only on positive authentication.

In another example embodiment, on successful authentication, an audio-video feed is made available between the patient 320-1 and the healthcare provider 320-2. The communication means as provided by the interface device is bonded with the communication from the apparatus and is exchanged via the channels.

In another example embodiment, on successful authentication, the system can provide for the healthcare provider to access historical data pertaining to the patient.

In another example embodiment, on successful authentication, the system can further provide for remote operation by the healthcare provider of the plurality of sensors coupled to the patient.

In another example embodiment, the operation of the apparatus and/or the docked/connected interface device, including the authentication and instruction to transmit data can be through audio commands either locally or remotely.

In another example implementation, the server can be configured to recognize instances when transmission of secure information can be allowed, and the server can be configured to allow flow of said secure information autonomously without explicitly requesting re-authorization from the patient.

In another example embodiment, the server can be configured with an Artificial Intelligence (AI) inference engine or interfaced with an external AI inference engine to process the patient vital sign data and the data related to the activities of daily living that are received from the patient's interface device system in order to infer various health and behavioral patterns and provide AI-based patient diagnostics and the treatment.

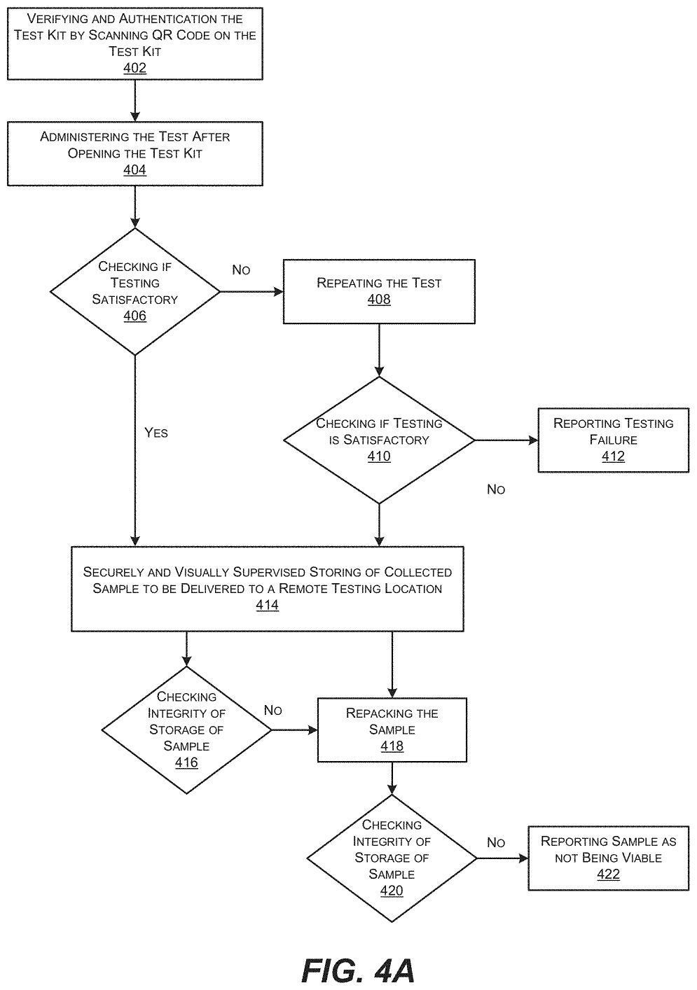

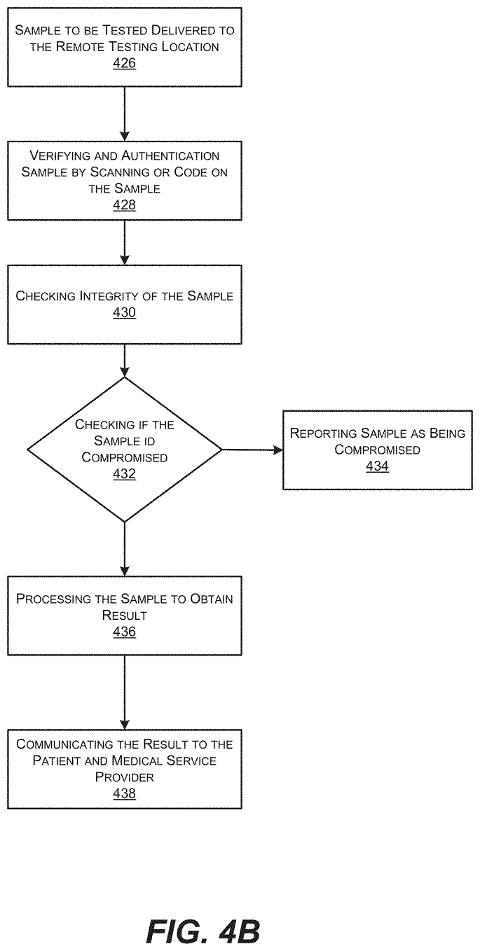

FIGS. 4A and 4B illustrate example flow diagrams for a process for facilitating remote diagnostic testing, in accordance with an embodiment of the present disclosure.

Referring to FIG. 4A, the method describes an approach to receive and administer the test at the location of the patient by the patient or a healthcare worker assigned to the patient.

In another example embodiment, the method may include, at 402, the step of verifying and authentication the SAT kit by scanning QR code on the SAT kit. The SAT kit can be delivered to the patient from an authorized vendor through a delivery service. An authentication pertaining to the SAT kit can be provided to the patient. The authentication code can be any such as QR code, bar code and other visual identification codes.

In an example embodiment of the present disclosure, the authentication code can be a QR code.

In another example embodiment, the QR code can include information pertaining to the SAT kit such as, but without limitations, the purpose of the testing kit, attributes of the testing kit such as date and place of manufacture of the testing kit, validity of the testing kit, the entity from whom the request is issued to procure the test (patient, or the medical service provider), identity of the patient, identity of the corresponding medical service provider, the identity of the apparatus at the location of the patient, and the identity of the apparatus at the location of the medical service provider. The above-mentioned information can be encoded into the QR code, the QR code being attached with the SAT kit. The QR code can also be a part of the tamper proof seal of the SAT kit, where, if the integrity of the seal is compromised, the QR can also be compromised.

In another example embodiment, a scanning device coupled to the apparatus can be adapted to scan the QR code of the testing kit, and the veracity of the testing kit can be authenticated by the apparatus. The authentication can include verification of the identity of the apparatus at the location of the patient and the identity of the patient, and the corresponding purpose of the testing kit.

In another example embodiment, the method may include, at 404 the step of opening the SAT kit. The apparatus at the locations of the patient and the medical service provider can be coupled, and the patient can be instructed to open the SAT kit, and, under guidance of the medical service provider, the patient can be instructed to administer the SAT.

In another example embodiment, the method may include, at 406, the step of checking of administering of test is done properly. In the event that the patient is unable to administer the test, a trained and authorized test administrator may be tasked with administering the test to the patient.

In an embodiment, the testing kit can be of the following types: instantaneous tests; and long-term tests.

In another example embodiment, instantaneous tests are those which, when administered, provide an immediate result. The results can then be communicated to the medical service provider through the apparatus, through secure and regulated communication channels.

In an example embodiment, if at 406, test was not satisfactory, then at 408, the method may include the step of repeating the test and then at 410 checking for satisfactory results. At 412, the method may include the step of reporting failure if at 410, the results are not satisfactory.

In another example embodiment, long-term tests are those which are required to be processed at a remote testing location such as a lab for the results to be derived. In such a case, the test is administered, and a sample of a material is collected and securely and visually supervised stored at step 414. At 416, the method may include the step of checking the integrity of the sample and at 418, the method may include repacking of the sample and then at 420, again checking the integrity of the storage of the sample. If the storage is not proper, then at 422, the method may include the step of reporting that the sample is not viable.

Referring to FIG. 4B, the method describes an approach to receive and test a collected sample from a patient at a remote testing location. In an embodiment, the sample is stored in one or more containers adapted for storage of the sample. The sample is packaged, and the package is sealed using a second QR code generated by the apparatus. The second QR code is encoded with information similar to the ones mentioned previously.

The samples are then delivered to the remote testing location via the delivery service at 426.

In another example embodiment, the sample received at the remote testing location and the sample may be verified and authenticated by scanning the QR code at 428 and then is similarly handled to check for integrity of the sample at 430. If at 432, the checking of the integrity of the sample is positive, then at 436, the sample is then processed. The results can then be communicated to the medical service provider and the patient through secure and regulated channels at 438. If at 432, the checking of the integrity of the sample is negative, then at 434, reporting that the sample is compromised.

While the above example only describes one of many tests that can be administered remotely under the supervision of a trusted authority, there can be many variations of such test administration procedures. The nature and the scope of the test may be different and more involved. The test may be even finetuning of the patient's explant or an implant which may require a special communicator involved between the apparatus of this invention and the patient's explant/implant. The examples of such communicators can be magnetic interrogator for the pacemaker implant or similarly for the DBS or sacral implant or EEG headgear for monitoring seizure activity of the patient. Hence the scope of the test and its remote administration can be different. The apparatus and the system of the present disclosure enable such extensions and are within the scope of the invention.

Generally, testing and calibration of patient implants and explants which are currently done by bringing the patient to the location of the medical healthcare provider. However, during times of spread of contagious illnesses, there is fear of infection, particularly at locations of medical healthcare providers such as hospitals and clinics. The present disclosure provides authenticated, supervised, interactive administration of treatment, tests and/or fine-tuning of the treatment/episode preventive equipment, which may be embedded or accompanied with the patient. While not each and every individual procedure is listed as part of the embodiments, the platform invention enables many variations of each of those procedures to accomplish the result under telemedicine and telehealth remote consultation providing better user experience for both the patient and the caregiver.

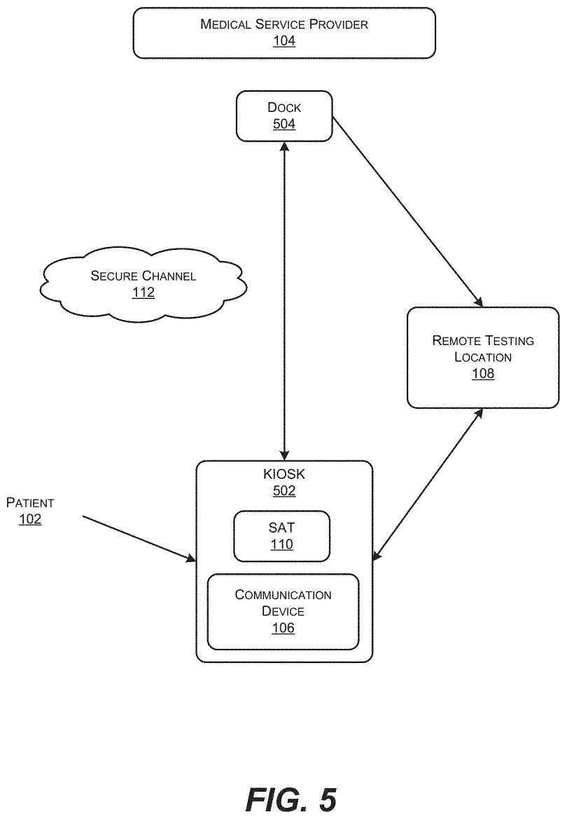

FIG. 5 illustrates an example representation of a system for facilitating remote diagnostic testing for a patient where the patient is required to visit a remote testing location, in accordance with an embodiment of the present disclosure. A remote testing location 108 can be any location or unit that can provide both instantaneous testing and long-term testing. The remote location can be situated in a same location where patient is, such as a dedicated room where tests are administered, or can be a remote unit.

In an example embodiment, the remote location can be a kiosk 502 or a mobile testing unit that can provide both instantaneous testing and long-term testing.

In an embodiment, SAT kits can be dispensed at the kiosk 502. SAT kits can be for different kinds of tests.

In another example embodiment, a patient 102 can request and procure a SAT kit of a specific configuration to suit the testing needs of the patient.

In another example embodiment, the SAT kit can be configured with a QR code with information including, but without limitations, the purpose of the testing kit and attributes of the testing kit such as date and place of manufacture of the testing kit.

In another example embodiment, the patient 102 is instructed to communicate audio-visually to a medical service provider using a communication means such as a smartphone or tablet.

In another example embodiment, the kiosk 502 or mobile testing unit can be provided with an apparatus which can be configured to couple with the communication means, through which the medical service provider and the patient can communicate. In another example embodiment, the apparatus can be adapted to scan the QR code of the testing kit, and the veracity of the testing kit can be authenticated by the apparatus.

In another example embodiment, the patient 102 can be expected to authenticate their identity by providing a proof of identity visually, through the apparatus.

The medical service provider 104 can instruct the patient to open the SAT kit, and, under guidance of the medical service provider, the patient can be instructed to administer the SAT.

In an embodiment, the testing kit can be a long-term test, in which case, a sample of a material of the patient is collected.

In an embodiment, the sample is stored in one or more containers adapted for storage of the sample. The sample is packaged, and the package is sealed using a second QR code generated by the apparatus. The second QR code is encoded with information similar to the ones mentioned previously. The samples are then delivered to the remote testing location via the delivery service.

In another example embodiment, the sample received at the remote testing location is similarly handled to check for integrity of the sample, and the sample is then processed. The results can then be communicated to the medical service provider and the patient through secure and regulated channels.

In another example embodiment, the test can be an instantaneous test. The results of the test can be communicated to the medical service provider through the apparatus, through secure and regulated communication channels.

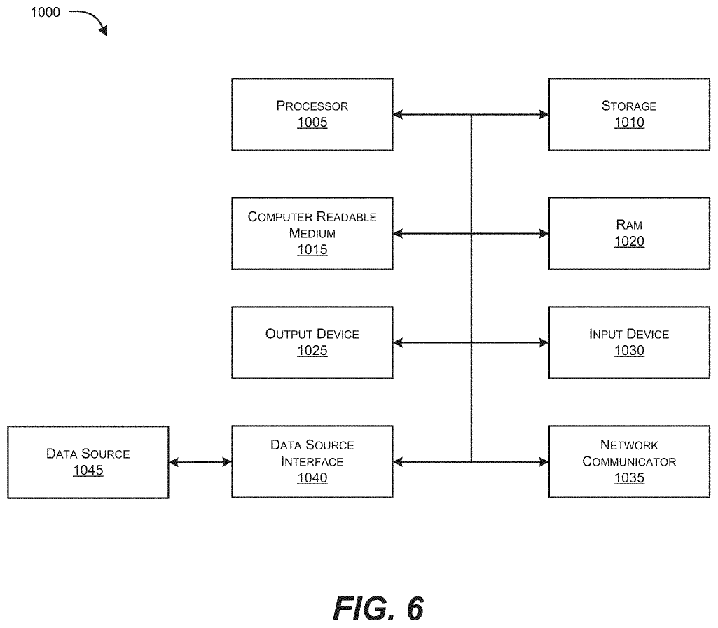

FIG. 6 illustrates a hardware platform 600 for implementation of the system 100, according to an example embodiment of the present disclosure. Particularly, computing machines such as but not limited to internal/external server clusters, quantum computers, desktops, laptops, smartphones, tablets and wearables which may be used to execute the system 100 or may have the structure of the hardware platform 600. The hardware platform 600 may include additional components not shown and that some of the components described may be removed and/or modified. In another example, a computer system with multiple GPUs can sit on external-cloud platforms including Amazon Web Services, Human Capital Management or internal corporate cloud computing clusters, or organizational computing resources, etc.

Over FIG. 6, the hardware platform 600 may be a computer system 600 that may be used with the examples described herein. The computer system 600 may represent a computational platform that includes components that may be in a server or another computer system. The computer system 600 may execute, by a processor (e.g., a single or multiple processors) or other hardware processing circuit, the methods, functions and other processes described herein. These methods, functions and other processes may be embodied as machine-readable instructions stored on a computer-readable medium, which may be non-transitory, such as hardware storage devices (e.g., RAM (random access memory), ROM (read-only memory), EPROM (erasable, programmable ROM), EEPROM (electrically erasable, programmable ROM), hard drives, and flash memory). The computer system 600 may include a processor 605 that executes software instructions or code stored on a non-transitory computer-readable storage medium 610 to perform methods of the present disclosure. The software code includes, for example, instructions to gather information pertaining risk factors and data elements in an environment and generate alerts, based on risk assessment of the environment. In an example, one or more of output of any of the data capturer 26, the data lake 18, the machine learning engine 24 may be software codes or components performing these steps.

The instructions on the computer-readable storage medium 610 are read and stored the instructions in storage 615 or in random access memory (RAM) 620. The storage 615 provides a large space for keeping static data where at least some instructions could be stored for later execution. The stored instructions may be further compiled to generate other representations of the instructions and dynamically stored in the RAM 620. The processor 605 reads instructions from the RAM 620 and performs actions as instructed.

The computer system 600 further includes an output device 625 to provide at least some of the results of the execution as output including, but not limited to, visual information to users, such as external agents. The output device can include a display on computing devices and virtual reality glasses. For example, the display can be a mobile phone screen or a laptop screen. GUIs and/or text are presented as an output on the display screen. The computer system 600 further includes input device 630 to provide a user or another device with mechanisms for entering data and/or otherwise interact with the computer system 600. The input device may include, for example, a keyboard, a keypad, a mouse, or a touchscreen. In an example, output of any of the data capturer 26, the data lake 18, the machine learning engine 24 may be displayed on the output device 625. Each of these output devices 625 and input devices 630 could be joined by one or more additional peripherals. In an example, the output device 625 may be used to provide alerts or display a risk assessment map of the environment.

A network communicator 635 may be provided to connect the computer system 600 to a network and in turn to other devices connected to the network including other clients, servers, data stores, and interfaces, for instance. A network communicator 635 may include, for example, a network adapter such as a LAN adapter or a wireless adapter. The computer system 600 includes a data source interface 640 to access data source 645. A data source is an information resource. As an example, a database of exceptions and rules may be a data source. Moreover, knowledge repositories and curated data may be other examples of data sources.

It can be appreciated by those versed in the art that the apparatus and system described herein are illustrations of an embodiment of the present disclosure and that they may not be construed as limitations to the scope of the present disclosure.

It should be apparent to those skilled in the art that many more modifications besides those already described are possible without departing from the inventive concepts herein. The inventive patent matter, therefore, is not to be restricted except in the spirit of the appended claims. Moreover, in interpreting both the specification and the claims, all terms should be interpreted in the broadest possible manner consistent with the context. In particular, the terms “includes” and “including” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc. The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

What is claimed is:

1. A self-administering testing system, the system comprising:
a processor;
a probe mechanism coupled to the processor, the probe mechanism to:
extract a sample comprising a body tissue of a first user, the body tissue pertaining to a site wherein medical examination is required for the first user;
a sensor module coupled to the processor, the sensor module to:
extract a set of signals pertaining to health conditions of a user;
a data capturer coupled to the processor to capture information pertaining to the first user;
a data lake coupled to the processor to store the captured information;
a machine learning engine coupled to the processor to:
examine the sample extracted from the probe mechanism;
extract a first set of attributes pertaining to the examined sample;
extract a second set of attributes from the set of signals extracted by the sensor module;
correlate the first and second set of attributes to determine a third set of attributes;
store the third set of attributes in the data lake; and
transmit the third set of attributes through a communication module associated with a first computing device associated with the first user and a second computing device associated with a second user.

2. The system as claimed in claim 1, wherein the system further comprises a verification and authentication module that verifies and authenticates the system based on a set of predefined instructions, wherein the verification and authentication module further checks expiration validity, type of the system, wherein a visual recognition mechanism is coupled to the authentication and verification module to visually recognize any or a combination of a predetermined pattern, timestamp for delivery and receipt the system, wherein the time stamp is recorded on the data lake.

3. The system as claimed in claim 1, wherein the system is configured to obtain a registration data based on a request from any unregistered first user and any unregistered second user through respective first computing device and second computing device, wherein login credentials are generated based on acknowledgement of a request and verification of the registration data, wherein the first user and the second user enter the generated login credentials to access the system to obtain the information service associated with the first user, wherein the first user is the patient and the second user is the health care provider.

4. The system as claimed in claim 3, wherein on successful authentication, the system further provides for remote operation by the second user of the sensor module coupled to the system, wherein the operation of the system, including the authentication and instruction to transmit information is through a set of audio, video or textual messages through the respective first computing device associated with the first user and the second computing device associated with the second user or through a communication module operatively coupled to the system.

5. The system as claimed in claim 1, wherein the communication module provides communication between the first computing device associated with the first user and the second computing device associated the second user adapted for visual and audio communications, wherein the communication module is configured with security protocols to facilitate communication between the first user and the second user.

6. The system as claimed in claim 1, wherein the sample extracted by the probe mechanism is collected and securely stored and sealed under supervision in one or more containers associated with the system, said one or more containers are marked with identification markers pertaining to identity of the first user, the nature of the test and the identity of the second user and the time stamp for administration of the system.

7. The system as claimed in claim 1, wherein a result associated with examination of the third set of attributes is provided in the form of a certificate indicating that the result provided is authenticated by a competent authority and wherein the certificate comprises the predetermined pattern that upon scanning by a scanner, a set of details included in the certificate is available to any user or entity performing the scan, wherein the set of details comprises, the information associated with the first user, any contagious disease suffering by the first user or a combination thereof.

8. A self-administering testing device, the device comprising:
a processor;
a probe mechanism coupled to the processor, the probe mechanism to:
extract a sample comprising a body tissue of a first user, the body tissue
pertaining to a site wherein medical examination is required for the first user;
a sensor module coupled to the processor, the sensor module to:
extract a set of signals pertaining to health conditions of a user;
a data capturer coupled to the processor to capture information pertaining to the first user;
a data lake coupled to the processor to store the captured information;
a machine learning engine coupled to the processor to:
examine the sample extracted from the probe mechanism;
extract a first set of attributes pertaining to the examined sample;
extract a second set of attributes from the set of signals extracted by the sensor module;
correlate the first and second set of attributes to determine a third set of attributes;
store the third set of attributes in the data lake;
transmit the third set of attributes through a communication module associated with a first computing device associated with the first user and a second computing device associated with a second user.

9. The device as claimed in claim 8, wherein the device further comprises a verification and authentication module that verifies and authenticates the device based on a set of predefined instructions, wherein the verification and authentication module further checks expiration validity, type of the device, wherein a visual recognition mechanism is coupled to the authentication and verification module to visually recognize any or a combination of a predetermined pattern, timestamp for delivery and receipt the device, wherein the time stamp is recorded on the data lake.

10. The device as claimed in claim 8, wherein a communication module provides communication between the first computing device associated with the first user and the second computing device associated the second user adapted for visual and audio communications, wherein the communication module is configured with security protocols to facilitate communication between the first user and the second user.

11. The device as claimed in claim 8, wherein the device is customized to suit the testing needs of the first user.

12. The device as claimed in claim 8, wherein the device is configured to communicate with a predetermined entity associated with providing and delivering the device.

13. The device as claimed in claim 8, wherein the device is configured with tamper-proof seals and weather-proof seals, and wherein the device is designed with a predefined size to fit inside and get delivered from a predetermined kiosk.

14. The device as claimed in claim 8, wherein the sample extracted by the probe mechanism is collected and securely stored and sealed under supervision in one or more containers associated with the device, said one or more containers are marked with identification markers pertaining to identity of the first user, the nature of the test and the identity of the second user and the time stamp for administration of the device.

15. The device as claimed in claim 8, wherein a result associated with examination of the third set of attributes is provided in the form of a certificate indicating that the result provided is authenticated by a competent authority and wherein the certificate comprises the predetermined pattern that upon scanning by a scanner, a set of details included in the certificate is available to any user or entity performing the scan, wherein the set of details comprises, the information associated with the first user, any contagious disease suffering by the first user or a combination thereof.

16. A non-transitory computer readable medium comprising machine executable instructions that are executable by a processor to:
examine a sample extracted from a probe mechanism, wherein the sample comprises a body tissue of a first user, the body tissue pertaining to a site wherein medical examination is required for the first user;
extract a first set of attributes pertaining to the examined sample;
extract a second set of attributes from a set of signals extracted by a sensor module;
correlate the first and second set of attributes to determine a third set of attributes;
store the third set of attributes in a data lake;
transmit the third set of attributes through a communication module associated with a first computing device associated with the first user and a second computing device associated with a second user.

17. The non-transitory computer readable medium as claimed in claim 16, upon determining, that the predictive analysis yields a negative response for the transformed data set, invalidate the executed machine learning model.

18. The non-transitory computer readable medium as claimed in claim 18, wherein a new machine learning model is tested with a sample dataset, trained with a training data set, and corresponding model results and performance evaluation metrics are validated by matching with a validation dataset, wherein the new model is maintained at the database.

19. The non-transitory computer readable medium as claimed in claim 16, wherein The non-transitory computer readable medium configures a verification and authentication module to verify and authenticate the device based on a set of predefined instructions, wherein the verification and authentication module further checks expiration validity, type of the device, wherein a visual recognition mechanism is coupled to the authentication and verification module to visually recognize any or a combination of a predetermined pattern, timestamp for delivery and receipt the device, wherein the time stamp is recorded on the data lake.

20. The non-transitory computer readable medium as claimed in claim 16, wherein the non-transitory computer readable medium configures a communication module to provide communication between the first computing device associated with the first user and the second computing device associated the second user adapted for visual and audio communications, wherein the non-transitory computer readable medium configures the communication module with security protocols to facilitate communication between the first user and the second user.
